(19) United States
(12) Patent Application Publication
(10) Pub. No.: US 2025/0223530 A1
(43) Pub. Date: Jul. 10, 2025

(54) SYSTEMS AND METHODS FOR PASSAGING ORGANOIDS

(71) Applicant: Molecular Devices (Austria) GmbH, Puch bei Hallein (AT)

(72) Inventor: Josef Atzler, Hallein (AT)

(21) Appl. No.: 18/847,138

(22) PCT Filed: Mar. 14, 2023
(86) PCT No.: PCT/IB2023/052478

§ 371 Date: Sep. 13, 2024

Related U.S. Application Data
(60) Provisional application No. 63/320,459, filed on Mar. 16, 2022.

(51) Int. Cl. C12M 3/00; C12M 1/00; C12M 1/32; C12M 3/06

(52) U.S. Cl. CPC C12M 21/08; C12M 23/12; C12M 23/16; C12M 23/34; C12M 23/40; C12M 29/00; C12M 47/12

(57) ABSTRACT

A microplate includes a cultivation well, a feeding well, and at least one separation channel there between. The cultivation well, feeding well, and the separation channel each contain a first solution, and the cultivation well contains a subject organoid. A method of using such a microplate includes sealingly engaging a pipette with the feeding well. At least some of the first solution is aspirated into the pipette while maintaining the seal between the pipette and the feeding well. This causes the aspirated portion of the first solution to flow from the cultivation well, through the separation channel, and into the feeding well. A second solution is injected from the pipette while maintaining the seal between the pipette and the feeding well. The injected second solution flows from the feeding well, through the at least one separation channel, and into the cultivation well.

### CROSS-REFERENCE TO RELATED APPLICATIONS

This application is the national phase of PCT International Patent Application No. PCT/IB2023/052478 filed on Mar. 14, 2023, which in turn claims priority to and the benefit of U.S. Provisional Application No. 63/320,459, filed on Mar. 16, 2022, the disclosure of which is hereby incorporated by reference in its entirety.

### BACKGROUND

An organoid is a collection of organ-specific cell types that develops from stem cells or organ progenitors, self-organizes through cell sorting, and spatially restricted lineage commitment in a manner similar to in vivo. Further, organoids exhibit several properties. These include having multiple organ-specific cell types, being capable of recapitulating some specific function of the organ, and having cells that group together and spatially organize. Organoids are formed by using stem cells or progenitor cells that are cultured in a 3D medium such as an extracellular matrix hydrogel, available commercially as Matrigel or Cultrex BME. Organoid bodies are made by embedding stem cells in the 3D medium. When pluripotent stem cells are used for the creation of the organoid, the cells are usually, but not always, allowed to form embryoid bodies. Those embryoid bodies are then pharmacologically treated with patterning factors to drive the formation of the desired organoid identity. Organoids have also been created using adult stem cells extracted from a target organ and cultured in 3D media. These processes are typically performed in vitro in microplates or well plates. The microplates or well plates are typically incubated, agitated, washed, aspirated, etc., under specific criteria to properly culture the organoid, while removing dead cells that are shed from the organoid as part of the culturing process.

### SUMMARY

In one aspect, the technology relates to a method of passaging a subject organoid in a microplate comprising a cultivation well, a feeding well, and at least one separation channel fluidically coupling the cultivation well and the feeding well, wherein the cultivation well, the feeding well, and the at least one separation channel each contain a first solution, and wherein the cultivation well contains the subject organoid, where the method includes: sealingly engaging a pipette with the feeding well; aspirating at least some of the first solution into the pipette while maintaining the seal between the pipette and the feeding well, wherein the aspirated portion of the first solution flows from the cultivation well, through the separation channel, and into the feeding well during aspiration; and injecting a second solution from the pipette while maintaining the seal between the pipette and the feeding well, wherein the injected second solution flows from the feeding well, through the at least one separation channel, and into the cultivation well during injection. In an example, the feeding well includes a feeding well axis and wherein the pipette comprises a pipette axis and wherein the feeding well axis and pipette axis are misaligned during the sealing engagement of the pipette with the feeding well. In another example, the method further includes disposing the microplate in a tilted position, and wherein sealingly engaging the pipette with the feeding well is performed while the microplate is in the tilted position. In another example, the first solution and the second solution are different. In yet another example, the first solution contains a first liquid constituent and at least one substantially dead cell released from the subject organoid. In still another example, sealingly engaging the pipette with the feeding well includes contacting the first solution with the pipette.

In another example of the above aspect, the method includes, prior to sealingly engaging the pipette with the feeding well, disposing a live cell into the cultivation well. In another example, the method includes, prior to aspirating at least some of the first solution into the pipette, introducing the first solution into the cultivation well, the separation channel, and the feeding well.

In another aspect, the technology relates to a microplate including: a body at least partially defining: a cultivation well including a cultivation well mouth and a cultivation well base; a feeding well including a feeding well mouth and a feeding well base, wherein the feeding well mouth includes a substantially round feeding well mouth cross section; and at least one separation channel communicatively coupling the cultivation well and the feeding well; and a sheet secured to the body, wherein the sheet at least partially defines the cultivation well, the feeding well, and the at least one separation channel. In an example, the feeding well base includes a feeding well base cross section different than the feeding well mouth cross section. In another example, the feeding well base cross section is substantially rectangular. In yet another example, the feeding well includes an intermediate cross section between feeding well mouth cross section and the feeding well base cross section that is different than both the feeding well mouth cross section and the feeding well base cross section. In still another example, the at least one separation channel comprises a plurality of separation channels.

In another example of the above aspect, cultivation well includes a plurality of cultivation wells and wherein the plurality of separation channels are communicatively coupled to at least two of the plurality of cultivation wells. In another example, the at least one separation channel has a maximum height dimension of about 50μ. In yet another example, the at least one separation channel has a plurality of sides, wherein at least one of the plurality of sides is the sheet. In still another example, the cultivation well is a plurality of cultivation wells and wherein the feeding well is a plurality of feeding wells.

In another example of the above aspect, the sheet is secured to the body at walls disposed between adjacent ones of the cultivation wells and the feeding wells. In another example, the body is a unitary part.

In another aspect, the technology relates to a microplate including: a plurality of cultivation wells; a plurality of feeding wells separated from the plurality of cultivation wells by a plurality of walls; means for forming a sealing engagement between at least one of the plurality of feeding wells and a pipette inserted into at least one of the plurality of feeding wells; at least one separation channel communicatively coupling a first one of the plurality of cultivation wells and a first one of the plurality of feeding wells; and a sheet secured to the plurality of walls, wherein the sheet at least partially defines the plurality of cultivation wells, the plurality of feeding wells, and the at least one separation channel.

### BRIEF DESCRIPTION OF THE DRAWINGS

The following drawing figures, which form a part of this application, are illustrative of described technology and are not meant to limit the scope of the disclosure as claimed in any manner, which scope shall be based on the claims appended hereto.

FIGS. 1A and 1B depict top and bottom perspective views of an organoid microplate.

FIGS. 2A and 2B depict partial section and enlarged partial section views of the organoid microplate of FIGS. 1A and 1B.

FIGS. 3A and 3B depict partial enlarged lower perspective and partial enlarged bottom views of the organoid microplate of FIGS. 1A and 1B.

FIGS. 4A and 4B depict section views of an organoid microplate with a pipette inserted into a feeding well thereof.

FIGS. 5A-5C depicts alternative sealing elements for use with organoid plates.

FIG. 6 depicts a method of passaging an organoid in an organoid plate.

### DETAILED DESCRIPTION

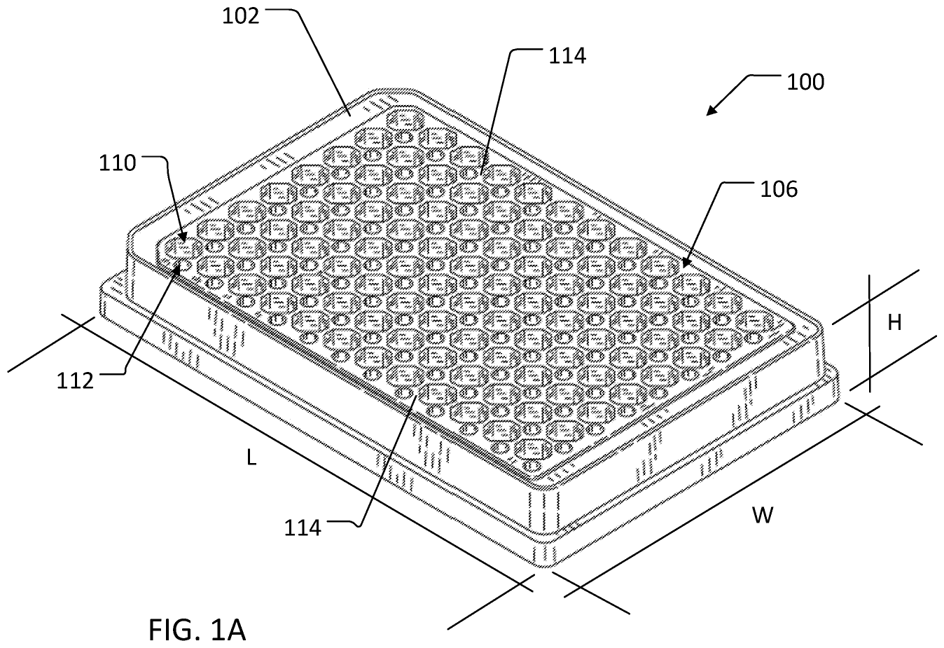

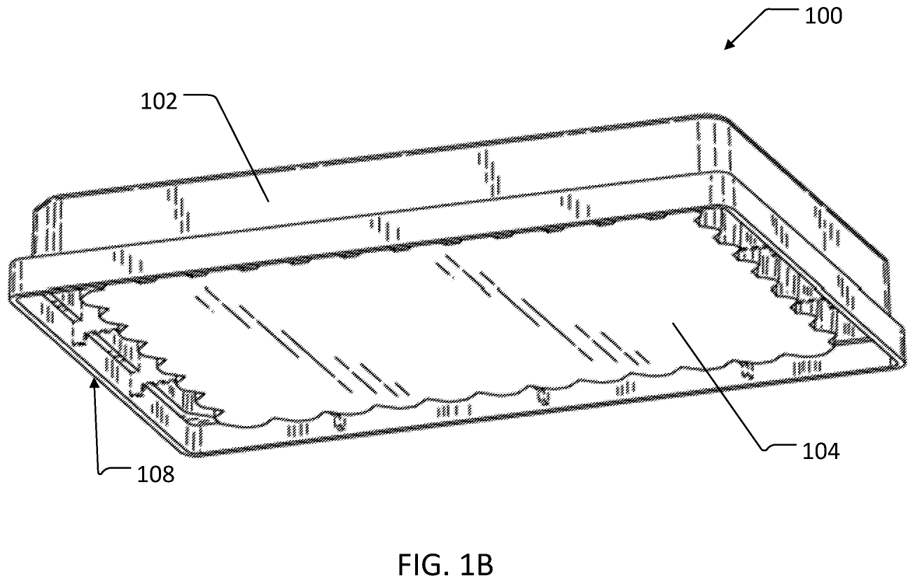

FIGS. 1A and 1B depict top and bottom perspective views of an organoid microplate 100 and are described concurrently. The microplate 100 (also described herein as a well plate) includes an upper portion or body 102 and a base 104 of a sheet material. As shown in FIG. 1, the upper portion includes a body 102 having a plurality of well units for growing, culturing, monitoring and assaying embryoid bodies, fused embryoid bodies, spheroids, organoids, and/or other multi-cellular bodies. As use herein, the term “organoid” is used generally to refer to cells or cell aggregates grown from stem cells or pluripotent stem cells within a microplate, regardless of the particular stage of growth or the specific type of cell or number thereof grown. In various examples, the well plate body 102 is made from a material having a topmost surface 106 and a bottommost surface 108. The microplate 100 may be characterized by a width W, length L, and height H. The components of the microplate body 102 may be formed of any suitable material by any suitable processes. In examples, the microplate body 102 may be formed exclusively or primarily of polymer, such as a transparent polymer, and/or other material as can be appreciated. For example, the polymer may include polystyrene, polypropylene, poly (methyl methacrylate), cyclic olefin polymer, cyclic olefin copolymer, and/or other polymer or polymers as can be appreciated. In examples, acrylonitrile butadiene styrene (ABS) may be utilized. The microplate body 102 may have no removable/moving parts, and/or may be formed as a single unitary piece, such as by injection molding or 3D resin printing, such that all of the structures (e.g., wells) of the microplate body 102 are formed integrally with one another. The base sheet material 104 may be secured to the bottom of the microplate body 102 as described herein, or may be formed integrally of the same material as the body 102. The materials used in the various components of the microplate 100 may be those compatible with each other, and that meet the required or desired performance characteristics of a microplate 100. The resin used for forming the body 102 of the microplate 100, as well as adhering the sheet material 104 to the bottom thereof, displays acceptable adhesion when cured, resistance to flow when uncured, and resistance to liquid seepage from the wells. In addition to the above listed materials, other materials used for the body 102 or sheet material 104 would be apparent to a person of skill in the art. For example, optically transparent foil may be used for the sheet material 104.

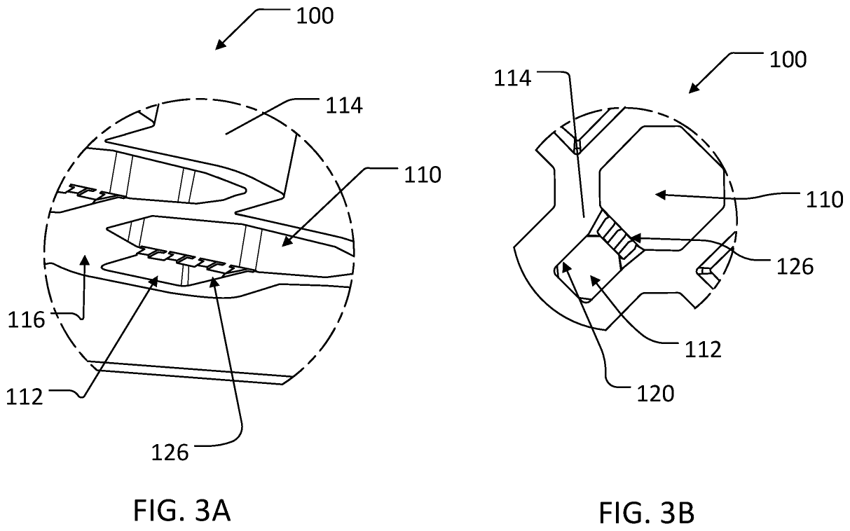

Each well unit comprises a primary well section, also referred to as a main, culture, or cultivation well 110, and a secondary well section, also referred to as a feeding or supply well 112. In various examples, the cultivation well 110 and the feeding well 112 can be fluidically connected with one another to facilitate a flow of liquid (e.g., feeding medium) between the cultivation well 110 and the feeding well 112, as described herein. For example, the cultivation well 110 and the feeding well 112 may be fluidically connected with one another via at least one separation channel (depicted below in FIGS. 3A and 3B) that is sized and shaped to facilitate the flow of a liquid media between the two well sections 110, 112. Exchanging the media between the cultivation well 110 and the feeding well 112 removes toxic by-products and supplies the growing cell cultures with fresh nutrients. As used herein, the term “well unit” refers to a fluidically connected combination of wells including at least one cultivation well 110 and at least one feeding well 112, coupled by at least one separation channel. In examples, multiple cultivation wells 110 may be fluidically coupled to and fed by a single feeding well 112. In other examples, multiple feeding wells 112 may be coupled to a single cultivation well 110. In still other examples, multiple cultivation wells 110 and multiple feeding wells 112 may be so coupled for liquid flow. As described in more detail below, the separation channels are formed in one or more walls 114 that generally separate adjacent wells from each other.

In examples, the cultivation wells 110 are sized and shaped to support deposited cell aggregates that may be embedded in hydrogel that is deposited in the base of each cultivation well 110. For example, each cultivation well 110 is used to grow the embryoid bodies, fused embryoid bodies, spheroids, organoids, and/or other multi-cellular bodies, as can be appreciated. According to various embodiments and dependent upon a number of well units in the microplate 100, the width of each cultivation well 110 can be up to about 8 millimeters (mm) (e.g., for 96 well plate), up to 11 mm (e.g., for a 48 well plate), up to about 17 mm (e.g., for a 24 well plate), and/or other sizes as can be appreciated. In addition, the depth of the cultivation wells 110 and the feeding wells 112 is specified such that the microplate 100 may be tilted as described below without spilling the liquid out of the respective cultivation wells 110 or feeding wells 112 of each the well units. In the example depicted in FIG. 1B, the depth of each of the cultivation wells 110 and feeding wells 112 is less than the total height H of the microplate 100, which may aid in stacking a plurality of microplates 100 for storage. As can be seen from FIG. 1B, the base sheet material 104 is positioned so as to be slightly higher than the bottommost surface 108 of the microplate 100.

The feeding wells 112 may be used to supply feeding media and/or other nutrients that can be used to feed the growing cell aggregates positioned in the cultivation wells 110. In addition, the feeding wells 112 can be used to harvest supernatant from the cell aggregates, as can be appreciated. For example, the feeding wells 112 may be used for the introduction of the feeding media and/or other nutrients that may be used by the growing cell culture in the cultivation wells 110. The feeding wells 112 are sized and shaped to hold liquid that can be exchanged with the cultivation wells 110 according to various embodiments of the present disclosure. According to various embodiments and dependent upon a number of well units in the microplate, the width of the feeding wells 112 can be up to about 8 millimeters (mm) (e.g., for 96 well plate), up to 11 mm (e.g., for a 48 well plate), up to about 17 mm (e.g., for a 24 well plate), and/or other sizes as can be appreciated.

The size and shape of the cultivation wells 110 and the feeding wells 112 may differ from one another. In some examples, the cultivation well 110 is larger (in a dimension, for example diameter or volume) than the feeding well 112. In other examples, the feeding well 112 is larger than the cultivation well 110. In some examples, the cultivation well 110 comprises a shape that differs from a shape of the feeding well 112. The well units are preferably arrayed in columns and rows as depicted in FIG. 1A. In various embodiments, the microplate 100 comprises a ninety-size (96) well-style plate comprising 96 primary well sections for cell cultures as can be appreciated. However, it should be noted that the microplate 100 is not limited to a 96 well-style plate and can be organized as a strip, or other type of configuration as can be appreciated. Additional details about the configuration of the feeding wells 112 are provided below.

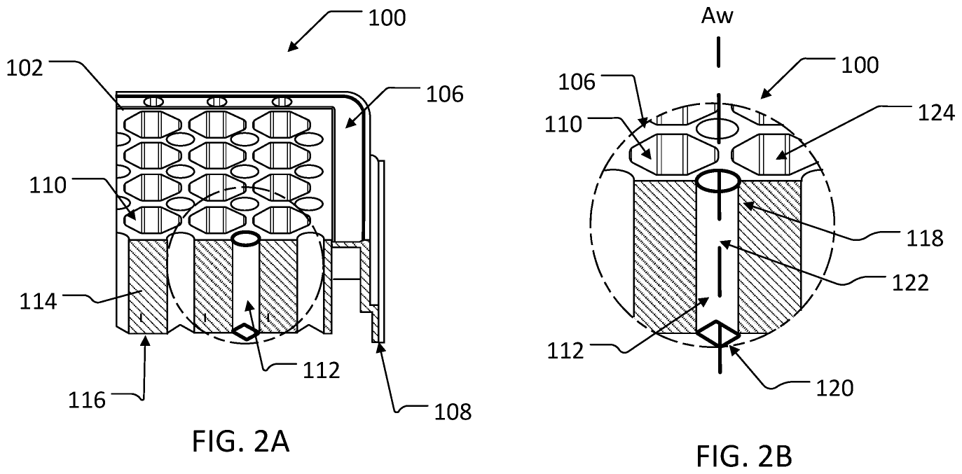

FIGS. 2A and 2B depict partial section and enlarged partial section views of the organoid microplate 100 of FIGS. 1A and 1B. Certain of the elements depicted in FIGS. 2A and 2B are described above in the context of FIGS. 1A and 1B and, as such, are not described further. FIGS. 2A and 2B are described concurrently and not every feature or element of one figure is depicted in another. The section views of FIGS. 2A and 2B generally expose an interior of the feeding channel 112. Further, a bottom edge 116 of the walls 114 is depicted and its elevated height relative to the bottommost surface 108 of the microplate 100 is apparent. The feeding wells 112 are formed in a particular configuration that enables particular performance. A number of advantages of the feeding well 112 configuration include ensuring a positive seal between the feeding well 112 and a pipette inserted therein, ensuring proper flow between the feeding well 112 and associated cultivation well 110, and ensuring unimpeded flow of liquids within the feeding well 112. The feeding well 112 may be defined by a feeding well axis Aw.

Each feeding well 112 includes a feeding well mouth 118 disposed proximate the uppermost surface 106 of the microplate 100. In examples, the feeding well mouth 118 comprises a substantially round cross section; in examples, the feeding well mouth 118 is circular, ovular, elliptical, or may be defined primarily by a curved perimeter. Further, the feeding well mouth 118 may be centered on the well axis Aw. The substantially round cross-sectional shape of the feeding well mouth 118 generally corresponds to an outer shape of a pipette (not shown), which enables sealing engagement of the feeding well 112 with a pipette when inserted therein, as described further below. In the depicted example, the feeding well 112 also includes a feeding well base 120 which has a cross sectional shape different than that of the feeding well mouth 118. The feeding well base 120 shape is substantially rectangular that is also substantially centered on the well axis Aw. In other examples, the shape may be one defined as substantially rectangular with rounded corners, a square, a squircle, or some other shape. A shape matching that of the feeding well mouth is also contemplated. A feeding well base 120 having at least one straight or substantially straight side may be desirable, however, to ensure desirable liquid flow through the separation channels, as described in more detail below. At a location 122 intermediate the feeding well mouth 118 and the feeding well base 120, a cross-sectional shape thereof may be different than that of both the feeding well mouth 118 and the feeding well base 120, as the cross-sectional shape of the feeding well 112 transitions from one to the other. It may be desirable for the walls of the feeding well 112 to smoothly transition from one shape to another, to avoid eddies or other features that may impede flow of liquid, debris from the cultivation well 110, etc. In FIG. 2B, cultivation well mouth 124 having a substantially hexagonal cross-sectional shape is depicted, although other shapes are contemplated.

FIGS. 3A and 3B depict partial enlarged lower perspective and partial enlarged bottom views of the organoid microplate 100 of FIGS. 1A and 1B, respectively. Certain of the elements depicted in FIGS. 3A and 3B are described above in the context of other figures and, as such, are not described further. FIGS. 3A and 3B are described concurrently and not every feature or element of one figure is depicted in another. A plurality of separation channels 126 are formed in the bottom edge 116 of a wall 114 that separates a cultivation well 110 of a well unit from an associated feeding well 112. In the depicted figures, four separation channels 126 are depicted, but a greater or fewer number of such channels 126 may be utilized. In cross section along the length of the separation channel 126, each may have a cross sectional profile of any desired or required shape, although substantially rectangular shapes may be desirable for maintaining desirable flows therethrough. In the depicted example, the material of the microplate body 102 forms an upper surface and two side surfaces of each separation channel 126. The sheet material (not shown) would define a bottom surface of each separation channel 126, once secured to the bottom edges 116 of the walls 114 of the body 102. Dimensions of the separation channels 126 may be as required or desired for a particular application. In examples, the height of the separation channels 126 may be about 10 μm, about 25 μm, or about 50 μm. In examples, heights of up to about 200 μm may be utilized for feeding large organoids in the cultivation well 110. Single cell applications may utilize separations channels 126 having heights less than about 10 μm, for example, less than about 5 μm or less than about 3 μm. The width of the separation channels 126 may also vary from application to application. For example, a single, wide separation channel may be utilized between the feeding well 112 and the cultivation well 110). Several separation channels 126 using a rectangular, quadratic, or round shape may be utilized where the width is no less than the height of the channel. The number of the channels may also vary. One, two, three, four, or more channels may be used, depending on the size of the wells and particular applications. The channels may be formed from the same molded plastic of the microplate 100. Alternatively, a very thin foil similar to the foil used to form the base of the microplate may be used to form the separation channels 126. In examples, a foil with 135 μm thickness may be utilized.

FIG. 3B more clearly depicts the cross-sectional shape of the base of the cultivation well 110 as substantially hexagonal. Other shapes are contemplated, but it may be advantageous to have a uniform or substantially uniform shape along the entire height of the cultivation well 110, to enable easy access to the cell aggregates therein, removal thereof from the well 110, etc. The substantially rectangular cross-sectional shape of the feeding well base 120 is also depicted. The separation channels 126 fluidically couple the two wells 110, 112, in this example, between substantially straight adjacent sides of the wells 110, 112. Although the separation channels 126 need not be identical in length (or shape), maintaining substantially similar dimensions may help ensure desirable liquid transfer between the wells 110, 112, without leaving residual liquid in one channel 126 while another channel 126 is drained to an empty condition.

The process of removing debris (e.g., dead cells or partial dead cells) from an organoid culture is referred to generally as passaging. With the microplate 100 depicted herein, as well as similarly configured microplates consistent with the teachings herein, passaging is performed by generating liquid flow between each cultivation well 110 and its coupled feeding well(s) 112. This may be required at multiple and particular times during an organoid forming process. By introducing and removing liquid from the feeding well 112, as opposed to directly from the cultivation well 110, several advantages are obtained. One advantage is that the separation channels 126 therebetween act as a filter to prevent live (and generally larger) cells and aggregates from being drawn out with the liquid and dead cells being removed. Another advantage is that inadvertent contact between the pipette and the live cells and organoids (and hydrogel) is eliminated, in that the pipette is not inserted into the cultivation well 110 until removal thereof is specifically required. Other advantages are described below.

In known microplates that include only cultivation wells (without the feeding wells described herein), passaging typically includes placing such a microplate in a centrifuge for a spinning operation. This operation compels organoids, large fractions, and live cells to a location in the cultivation well 110 separate from the dead cells, which will form a top layer within the liquid in the cultivation well, or may otherwise be suspended in the liquid. This top layer of liquid including debris (e.g., dead cells), must then be removed, followed by introduction of a new, clean liquid, if appropriate in the process. Insertion of the pipette into the cultivation cell for such removal requires fine positioning of the pipette to prevent inadvertent removal of the organoid or other desirable cells or aggregates.

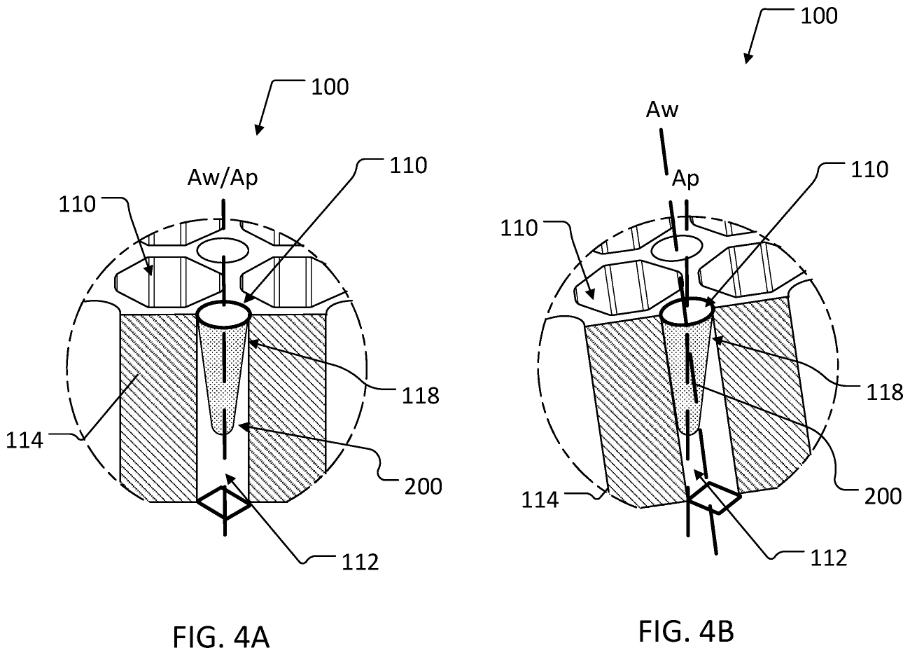

The microplates described herein, along with microplates manufactured consistent with the teachings of this specification, however, include a feeding well that may be used to reduce inadvertent removal of organoids and otherwise improve organoid forming processes. Instead of placing the microplates described herein in a centrifuge, in examples, the present technology also makes use of gravity for separation. Returning to the figures described above, the microplate 100 may be tipped or tilted, causing the organoids and heavier live cells and fractions to collect away from the separation channels 126. A pipette is then inserted into the feeding well 112 and the liquid therein aspirated therefrom. This aspiration draws liquid from the cultivation well 110, through the separation channels 126, into the feeding well 112, and into the pipette. Lighter dead cells and other debris suspended in the liquid is removed therewith, while the heaver organoids, live cells, and aggregates remain, since the separation channels 126 act as a filter to such larger products. New liquids or other solutions may then be introduced into the feeding wells 112 and may flow by gravity to a condition of equilibrium with the cultivation well 110. This gravitational flow, however, may be undesirably slow for certain applications. In examples, due to the dimensions and number of the separation channels, liquid introduced by gravity to the feeding well 112 may take up to 1 minute to flow into the cultivation well 110 to achieve a state of equilibrium. The particular structural features of the microplate 100 described herein, as well as similar configurations as would be apparent to a person of skill in the art, further improve aspiration and injection processes performed as part of organoid passaging. Those processes are described below initially in the context of FIGS. 4A and 4B.

FIGS. 4A and 4B depict section views of an organoid microplate 100 with a pipette 200 inserted into a feeding well 112 thereof. The pipette 200 is inserted into the feeding well 112 and may penetrate to a depth that the pipette 200 contacts liquid that may be present in the feeding well 112. The contact, however, is not required for aspiration or injection of liquid. This is because the feeding tube mouth 118 is substantially round and therefore forms a sealing engagement with the pipette 200 itself, which is also substantially round. Because of this sealing engagement, during aspiration, liquid is drawn from the cultivation well 110, through the separation channels 126, into the feeding well 112, and into the pipette, which is under a negative pressure. When injecting liquid, the sealing engagement enables positive pressure introduction of the liquid from the pipette, and therefore passage of the liquid into the feeding well 112, through the separation channels 126, and into the cultivation well 110. In examples, flow rates of up to about 80 μL/sec, up to about 90 μL/sec, up to about 95 μL/sec, up to about 100 μL/sec, up to about 105 μL/sec, and up to about 110 μL/sec may be achieved, which greatly reducing operational time. In FIG. 4A, the well axis Aw and a pipette Ap may be substantially aligned, as depicted.

The configuration of the microplate 100, with the substantially round feeding well mouths 118 that sealing engage with the pipette, allows for further advantages, one of which is depicted in FIG. 4B. Here, the microplate 100 has been tilted as part of the organoid passaging process as described above. The sealing engagement between the substantially round feeding well mouth 118 and the substantially round pipette 200 may be maintained during tilting of the microplate plate, for example at tilt angles up to about 2°, up to about 3°, up to about 4°, up to about 5°, up to about 6°, up to about 7°, or higher. Under these conditions, the well axis Aw and a pipette Ap are misaligned, as depicted in FIG. 4B. This may be particularly advantageous because aspiration while maintaining a tilt of the microplate 100 may help keep the organoids and live cells away from the separation channels, reducing inadvertent aspiration thereof. Further, it should be noted that automated pipette systems that include a plurality of pipettes, generally in a row, are mechanically actuated (raised and lowered), typically only vertically. Thus, the microplates 100 such as described herein, that utilize structures that enable sealing engagement with pipettes, may still be utilized with such automated systems, even while the microplate 100 remains in a tilted configuration.

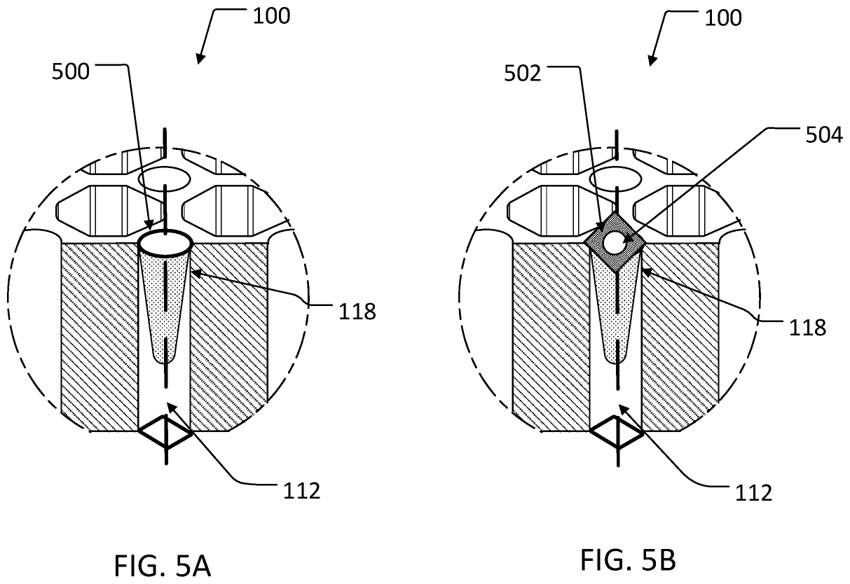

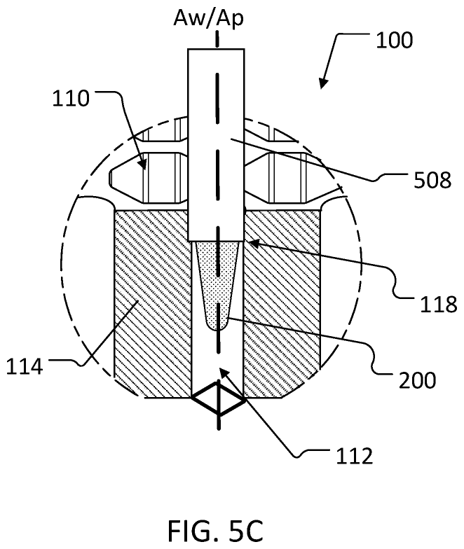

FIGS. 5A-5C depict alternative sealing elements for use with organoid microplates 100. In general, each of these sealing elements are used to sealingly engage a pipette with the feeding well 112 of a microplate 100. In the depicted examples, the sealing elements are disposed adjacent the feeding well mouth 118 during aspiration or injection processes. FIG. 5A depicts a sealing element that includes a gasket 500, which may be secured proximate a perimeter of the feeding well mouth 118, for example, with an adhesive or other fastener. In other examples, the gasket may instead be secured to the pipette and contact the feeding well mouth 118 to form a sealing engagement. FIG. 5B depicts an elastic or flexible septum 502, which may span the entire feeding well mouth 118. The septum 502 may define an opening 504 of sufficient diameter to receive and close around an end of the pipette 200 when inserted. An arrangement of septa sufficient to cover the upper surface of a microplate 100 may be formed in a single sheet, with appropriate openings 504, as well as openings to allow access to the cultivation wells, formed therein. That sheet may then be adhered to the upper surface of the microplate 100.

FIG. 5C depicts an elongate throat 508 that may extend from the feeding well mouth 118. The throat 508 may extend above an upper surface and be tapered or otherwise shaped to match the taper of the pipette 200. The taper need not be an exact match but may merely be sufficient to provide contact with the outer surface of the pipette 200 adequate to form a sealing engagement once inserted. Another example of a sealing element contemplates an upper portion of a microplate being formed of a material more flexible or resilient than the remainder of the microplate, such that that portion will more easily conform to the pipette.

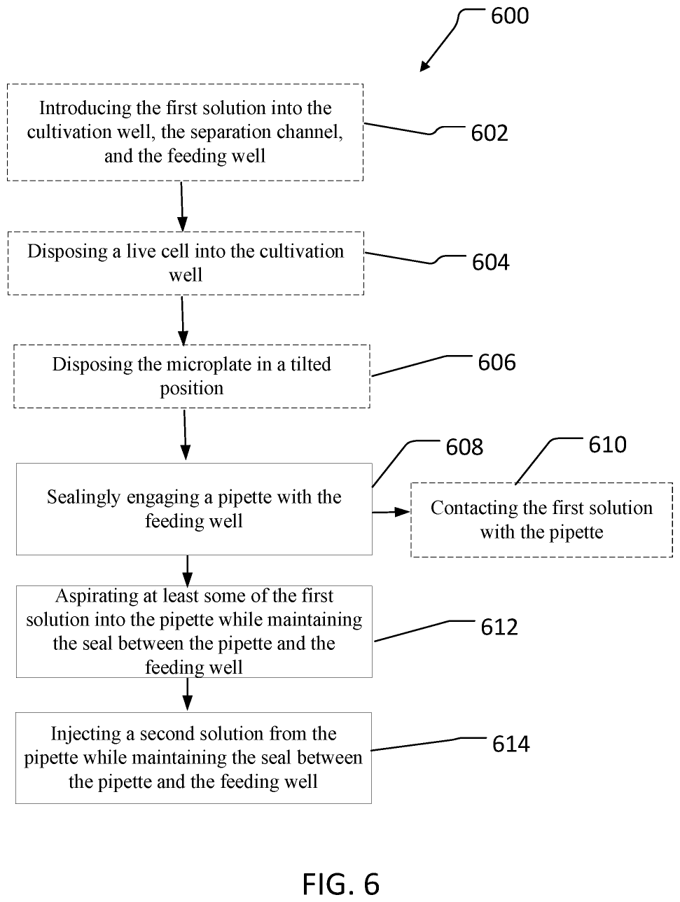

FIG. 6 depicts a method 600 of passaging a subject organoid in a microplate. The microplate may be configured as described herein or may be otherwise consistent with the teachings herein, a would be apparent to a person of skill in the art upon reading the entire disclosure. In that regard, the microplate may include at least one cultivation well, at least one feeding well, and at least one separation channel fluidically coupling the cultivation well and the feeding well. In many applications, a plurality of cultivation wells fluidically coupled to a plurality of feeding wells via a plurality of separation channels will be included in the microplate. Further, a matrix such as hydrogel may be disposed in the cultivation well to support the live cells, organoids, etc., during the forming process. The method 600 may begin with two optional operations. In operation 602, a first liquid solution is introduced into the cultivation well, the separation channel, and the feeding well. This liquid solution may be introduced into the feeding well or the cultivation well until a desired liquid equilibrium is attained. In optional operation 604, a live cell may be disposed into the cultivation well, onto the matrix, via known methods. Operations 602 and 604 may be performed in the order depicted, in reserve order, or substantially simultaneously. At some point during the organoid forming process, the microplate may be tilted, operation 606, to collect the desirable contents (e.g., live cells, organoids, etc.) in a location away from the separation channels.

The method 600 continues to operation 608, sealingly engaging a pipette with the feeding well. As described elsewhere herein, sealing engagement may occur when the pipette contacts the feeding well mouth so as to form a seal. Other examples are also described herein, such as a seal being formed by contact with a gasket, a septum, or an elongate throat shaped to sealingly mate with the pipette. If optional operation 606 has been performed, a feeding well axis that defines the feeding well will be misaligned with a pipette axis that defines the pipette during sealing engagement thereof. Depending on the height of liquid disposed in the feeding well, length and other dimensions of the feeding well, and depth of insertion of the pipette into the feeding well, the first solution may be contacted as the sealing engagement is made, operation 610. Further, operations 608 and 610 may be performed substantially simultaneously with operation 606, where the microplate is maintained in a tilted position during sealing engagement of the pipette with the feeding well, so as to prevent inadvertent aspiration of organoids, live cells, etc., during subsequent operations.

While sealing engagement is maintained, operation 612 is performed. There, at least some of the first solution is aspirated into the pipette. During this aspiration, the first solution flows from the cultivation well, through the separation channel, and into the feeding well during aspiration, so as to be drawn into the pipette. In examples, the aspirated first solution may contain a first liquid constituent and at least one substantially dead cell released from the subject organoid. At this point, the pipette may be removed from sealing engagement with the feeding well, so as to enable disposal of the aspirated first solution. Thereafter, in operation 614, a second solution may be injected from the pipette while maintaining the seal between the pipette and the feeding well. The first solution and the second solution may be different. In other examples, the second solution may be the same as the first solution, which may cause a turbulent agitation of the solution, to help release debris such as dead cells from the organoid. Once sufficient agitation is performed, the agitated solution and contents thereof may be aspirated and disposed of. The injected second solution flows from the feeding well, through the at least one separation channel, and into the cultivation well during injection. The pipette used for the injection operation may be the same pipette as used for the aspiration operation, or a different pipette may be used, consistent with best practices for a laboratory in which the method 600 is performed, e.g., to prevent cross-contamination. Thus, in the context of the method 600 described herein, the term “the pipette” should not be considered limiting to a single, same pipette. In examples, the aspirated first solution may contain a first liquid constituent and at least one substantially dead cell released from the subject organoid.

It is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples only and is not intended to be limiting. It must be noted that, as used in this specification, the singular forms “a,” “an,” and “the” include plural referents unless the context clearly dictates otherwise.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified examples and examples. In this regard, any number of the features of the different examples described herein may be combined into one single example and alternate examples having fewer than or more than all of the features herein described are possible.

While various examples have been described for purposes of this disclosure, various changes and modifications may be made which are well within the scope contemplated by the present disclosure. Numerous other changes may be made which will readily suggest themselves to those skilled in the art and which are encompassed in the spirit of the disclosure.

1. A method of passaging a subject organoid in a microplate comprising a cultivation well, a feeding well, and at least one separation channel fluidically coupling the cultivation well and the feeding well, wherein the cultivation well, the feeding well, and the at least one separation channel each contain a first solution, and wherein the cultivation well contains the subject organoid, the method comprising:
sealingly engaging a pipette with the feeding well;
aspirating at least some of the first solution into the pipette while maintaining the seal between the pipette and the feeding well, wherein the aspirated portion of the first solution flows from the cultivation well, through the separation channel, and into the feeding well during aspiration; and
injecting a second solution from the pipette while maintaining the seal between the pipette and the feeding well, wherein the injected second solution flows from the feeding well, through the at least one separation channel, and into the cultivation well during injection.

2. The method of claim 1, wherein the feeding well comprises a feeding well axis and wherein the pipette comprises a pipette axis and wherein the feeding well axis and pipette axis are misaligned during the sealing engagement of the pipette with the feeding well.

3. The method of claim 2, further comprising disposing the microplate in a tilted position, and wherein sealingly engaging the pipette with the feeding well is performed while the microplate is in the tilted position.

4. The method of claim 1, wherein the first solution and the second solution are different.

5. The method of claim 4, wherein the first solution contains a first liquid constituent and at least one substantially dead cell released from the subject organoid.

6. The method of claim 1, wherein sealingly engaging the pipette with the feeding well comprises contacting the first solution with the pipette.

7. The method of claim 1, further comprising, prior to sealingly engaging the pipette with the feeding well, disposing a live cell into the cultivation well.

8. The method of claim 7, further comprising, prior to aspirating at least some of the first solution into the pipette, introducing the first solution into the cultivation well, the separation channel, and the feeding well.

9. A microplate comprising:
a body at least partially defining:
a cultivation well comprising a cultivation well mouth and a cultivation well base;
a feeding well comprising a feeding well mouth and a feeding well base, wherein the feeding well mouth comprises a substantially round feeding well mouth cross section; and
at least one separation channel communicatively coupling the cultivation well and the feeding well; and
a sheet secured to the body, wherein the sheet at least partially defines the cultivation well, the feeding well, and the at least one separation channel.

10. The microplate of claim 9, wherein the feeding well base comprises a feeding well base cross section different than the feeding well mouth cross section.

11. The microplate of claim 10, wherein the feeding well base cross section is substantially rectangular.

12. The microplate of claim 10, wherein the feeding well comprises an intermediate cross section between feeding well mouth cross section and the feeding well base cross section that is different than both the feeding well mouth cross section and the feeding well base cross section.

13. The microplate of claim 9, wherein the at least one separation channel comprises a plurality of separation channels.

14. The microplate of claim 13, wherein the cultivation well comprises a plurality of cultivation wells and wherein the plurality of separation channels are communicatively coupled to at least two of the plurality of cultivation wells.

15. The microplate of claim 9, wherein the at least one separation channel comprises a maximum height dimension of about 50μ.

16. The microplate of claim 9, wherein the at least one separation channel comprises a plurality of sides, wherein at least one of the plurality of sides comprises the sheet.

17. The microplate of claim 9, wherein the cultivation well comprises a plurality of cultivation wells and wherein the feeding well comprises a plurality of feeding wells.

18. The microplate of claim 17, wherein the sheet is secured to the body at walls disposed between adjacent ones of the cultivation wells and the feeding wells.

19. The microplate of claim 9, wherein the body comprises a unitary part.

20. A microplate comprising:
a plurality of cultivation wells;
a plurality of feeding wells separated from the plurality of cultivation wells by a plurality of walls;
means for forming a sealing engagement between at least one of the plurality of feeding wells and a pipette inserted into at least one of the plurality of feeding wells;
at least one separation channel communicatively coupling a first one of the plurality of cultivation wells and a first one of the plurality of feeding wells; and
a sheet secured to the plurality of walls, wherein the sheet at least partially defines the plurality of cultivation wells, the plurality of feeding wells, and the at least one separation channel.
